(19) United States
(12) Patent Application Publication
(10) Pub. No.: US 2022/0246285 A1
(43) Pub. Date: Aug. 4, 2022

(54) HOSPITAL CURTAIN PROTOCOL AND TRACKING SYSTEM

(71) Applicant: KleenEdge, LLC, Seattle, WA (US)

(72) Inventor: Karen Goelst, Seattle, WA (US)

(73) Assignee: KleenEdge, LLC, Seattle, WA (US)

(21) Appl. No.: 17/167,397

(22) Filed: Feb. 4, 2021

(51) Int. Cl. G16H 40/20; G16H 70/20; G16H 15/00; A47K 3/38

(52) U.S. Cl. CPC G16H 40/20; G16H 70/20; G06K 7/1413; A47K 3/38; G16H 15/00

(57) ABSTRACT

A system and method for customizing protocols associated with curtains in a hospital is disclosed. The method may include receiving, by the processor, a scan of a wall tag associated with a location; providing, by the processor, a list of installed curtains in the location; receiving, by the processor, a replacement reason for one or more of the installed curtains; and receiving, by the processor, a scan of a curtain tag for a new curtain.

### TECHNICAL FIELD

This disclosure generally relates to a protocol, inventory and tracking system, and more particularly, to a system and method for setting exchange protocols and tracking the replacement cycle for hospital curtains.

### BACKGROUND

The U.S. healthcare industry reports that they experience 4.9 million healthcare associated infections (HAIs) resulting in 480,000 U.S. patient deaths per year. Hospital curtains are high risk touchpoints for HAIs because many people in hospitals touch the curtains and/or contaminants come in contact with the curtains. After just two weeks in a patient's room, the curtains may be contaminated with MRSA, C.diff and VRE.

Despite being a high risk for HAIs, hospital curtains are infrequently cleaned, and many hospitals only exchange their curtains once per year. The infrequent curtain changes are often due to exchange costs, laundry costs, product costs and documentation costs. Hospital curtains hang from the ceiling, so staff must typically obtain, set-up, use and break down tall ladders to remove the curtains. In fact, the curtain removal process may take 45 minutes per room. After removing the curtains, staff must then bring the curtains to be cleaned. However, the laundry, tracking and re-hanging processes are challenging in that many curtains are different sizes, inventory constraints exist and large specialized laundry machines are needed. Moreover, about 10% of the curtains may be lost or damaged during the removal, laundry and re-hanging process, so those curtains may need to be replaced resulting in additional expenses. Furthermore, most hospitals do not have sufficient data or reminders about the curtains such that the hospitals often rely on handwritten logs with expiration dates handwritten on the labels of each of the curtains.

Therefore, a long-felt need exists for a curtain protocol compliance, inventory and tracking system that mitigates HAI risk, analyzes the risk of ISO (isolation exchanges) violations when determining relevant exchange frequencies, eliminates manual documentation, validates compliance, provides a data-driven solution and reduces or eliminates the aforementioned challenges associated with the curtains in existing hospital systems.

### SUMMARY

A system and method for customizing protocols associated with curtains in a hospital is disclosed. The method may include receiving, by the processor, a scan of a wall tag associated with a location; providing, by the processor, a list of installed curtains in the location; receiving, by the processor, a replacement reason for one or more of the installed curtains; and receiving, by the processor, a scan of a curtain tag for a new curtain.

The method may further include providing a replace all notification, in response to receiving a Covid-19 reason for removal. The method may also include receiving, by the processor, a work order request; and providing, by the processor, a list of scheduled curtain exchanges. The method may also include providing a notification that the new curtain cannot be installed, in response to the new curtain being from a previous location with a health concern. The method may also include receiving, by the processor, a scan of the curtain tag for the installed curtain that is being removed, in response to the installed curtain being soiled. The method may also include providing, by the processor, an option to replace all or replace some, in response to another reason for replacing the installed curtain. The method may also include receiving, by the processor, an install command, in response to a curtain being added without the curtain tag being scanned. The method may also include receiving, by the processor, a remove command, in response to a room being blocked for ISO and no curtains remain at the location. The method may also include receiving, by the processor, a remove command, in response to changing the installed curtains to a different curtain type at the location.

The replacement reasons may include at least one of ISO, Covid-19, Soiled or Other. The replacement protocol may be associated with each of the installed curtains. The replacement protocol may be associated with each of the installed curtains, wherein the replacement protocol is at least one of per patient, weekly, monthly, quarterly, bi-annually or annually.

The method may also include providing, by the processor, a report about curtain exchanges. The method may also include providing, by the processor, a report about overdue, upcoming, ISO trending or historical curtain exchanges.

### BRIEF DESCRIPTION OF THE DRAWINGS

The accompanying drawings, wherein like numerals depict like elements, illustrate exemplary embodiments of the present disclosure, and together with the description, serve to explain the principles of the disclosure. In the drawings:

FIG. 1 is an exemplary user interface that monitors multiple items, provides a description of each item and provides a removal reason, in accordance with various embodiments.

FIG. 2 is an exemplary user interface that provides location information, status information and protocol information for each product assigned to the location, in accordance with various embodiments.

FIG. 3 is an exemplary user interface that shows tag information, in accordance with various embodiments.

FIG. 4 is an exemplary dashboard, in accordance with various embodiments.

### DETAILED DESCRIPTION

In various embodiments, the system includes software that may be in the form of a web portal and/or an app. The system may include a PHP and/or MySQL application. The web app may support PC, Macintosh, Android tablets, iPads and/or mobile phones. The system may be accessed by any of those devices. The software may include scanning functionality that interacts with tags. The scanned tag data may be transmitted to the web portal in real-time. The system may eliminate all (or any portion of) manual documentation. The system may also reduce labor time more than 95%. The system (e.g., app) may be used across multiple platforms while using the same tag.

While the system may be described with respect to managing the inventory of curtains and tracking curtains, one skilled in the art will appreciate that the system may be similarly applied to any product or object. For example, the system may be used for furniture, bed sheets, pillows, mattresses, protective gear, surgical equipment, medicines, computer equipment, communication equipment, telephones and/or the like.

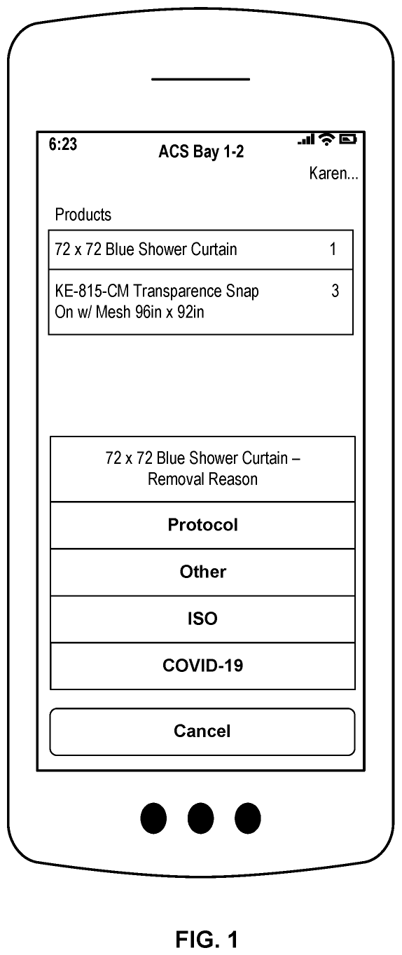

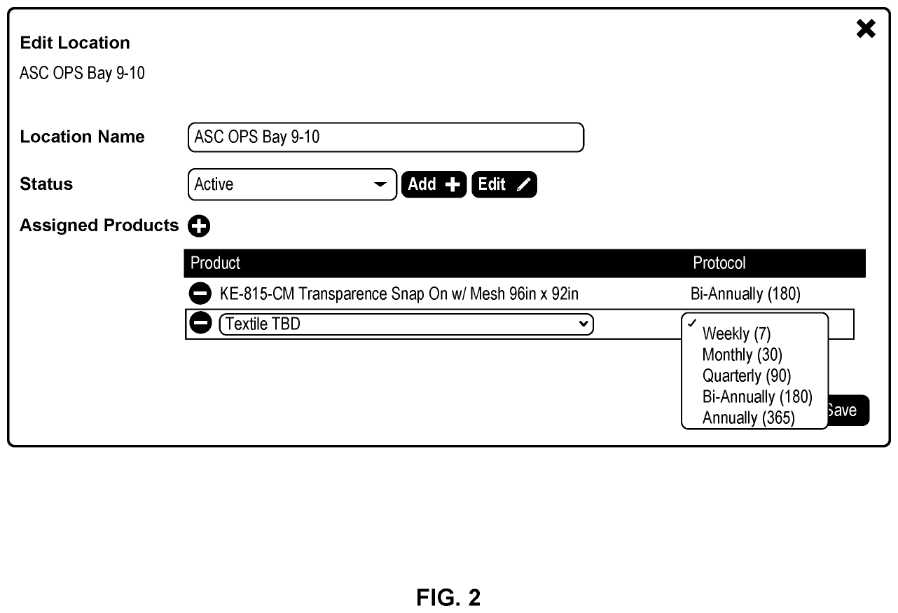

In various embodiments, the system allows for setting and/or customizing a protocol. The system also allows for setting and/or customizing an alert. The system may set multiple protocols or custom protocols for a particular location. The system may allow the setting of protocols per product. For example, a cubicle curtain is set to be replaced monthly, a detachable mesh (that may snap to the top of a curtain) may be set to be replaced annually and a roller shade for a room may be set to be replaced annually. The protocols may include curtain replacement guidelines per patient, weekly, monthly, quarterly, bi-annually and/or annually. For example, the system may set a protocol that an intensive care unit (ICU) should have curtains replaced weekly, an emergency room should have curtains replaced monthly, and a long-term stay hospital room should have curtains replaced quarterly or after a patient checks out of the room. As set forth in FIG. 1, the system also allows for the inclusion of a reason why a curtain was removed. The reason may be, for example, an ISO, COVID-19, protocol, soiled curtains, add a curtain or other reason. As set forth in FIG. 2, the system may also provide a location information such as, for example, room number, room name, bed location, bay information, etc. The system may set or block a location based on room status. For example, to protect staff, the system may block the replacement of curtains in high risk rooms (e.g., a room that currently includes a Covid-19 patient).

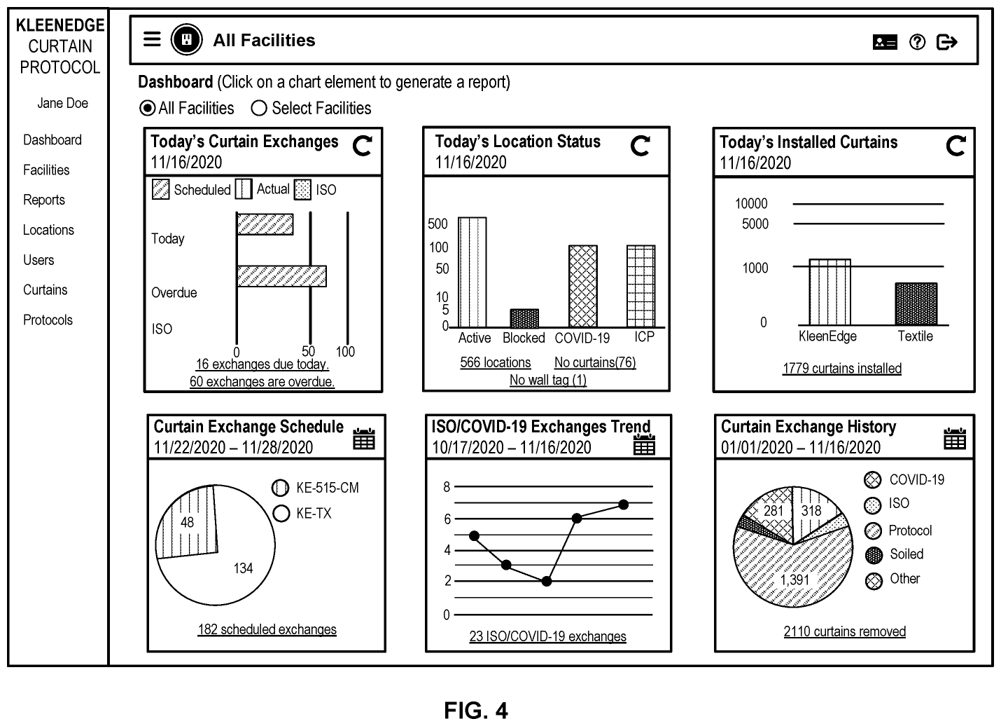

As set forth in FIG. 4, in various embodiments, the system may aggregate scanned and input data. The system may use the data to provide reporting and/or dashboard functions and analytics that may provide an overview of curtain exchange activities. The reports may be corporate-wide, customer-level, facility level, floor-level or any other subset. The dashboard may provide real time monitoring of current, upcoming, ISO trending, historical and/or overdue curtain exchanges. The dashboard may also provide contaminant (e.g., ISO, Covid-19, etc.) exchange tracking. The reports may provide insights about curtain data such as data about being overdue, upcoming, ISO trending and/or historical. The reports may provide information about the number of exchanges that are due today and the number of exchanges that are overdue. Such data about exchanges may be divided into the reasons for the exchanges such as scheduled exchanges, actual exchanges completed and ISO.

With respect to ISO, instead of using “blanket protocols” that are similar for all rooms or areas, the protocol software empowers facilities to customize protocols per area dependent on risk. For example, the ER or ICU may have a more frequent changing cycle compared to a patient room or a changing room. The system and reports allows the facility to subsequently monitor ISO exchanges through “ISO trending,” thus enabling the facility to analyze whether the facility is able to secure better outcomes (e.g., lowering HAI percentages) from more frequent curtain exchanges. As such, the system allows facilities to make more “evidence based” decisions about protocols and curtain replacements.

As used herein, ISO relates to “isolation exchanges” which may include curtain exchanges that take place when a room is considered “infected”. At such time, the facility may go into a “terminal clean” mode, wherein items are disinfected. For example, most items may be scrubbed down with Clorox and the curtains are exchanged.

The system includes an “articulation” function that sorts the curtain replacements by reasons for removal. Such function may provide a list of all curtains that were replaced for a certain reason. For example, the system may provide a list of all curtains that were replaced due to Covid-19 related issues for a hospital to help track Covid-19 related expenses. The data may include information about how many locations have curtains, how many locations do not have curtains, how many locations have wall tags, how many locations do not have wall tags, number of curtains installed, type of curtains installed, number of scheduled exchanges, types of curtains being exchanged, trends of exchanges, number of curtains exchanged for each of different reasons over a certain time period, etc. The location status may include active, blocked, Covid-19, etc. As discussed herein, the system may provide data about Covid-19, but one skilled in the art will appreciate that the system may provide similar information about any contaminant, disease, virus, pathogen, bacteria, mold, etc.

In various embodiments, by selecting the “facilities” option, the system provides a quick overview of the exchange status for the facilities. For example, for a particular hospital chain having many facilities, the system displays each facility location along with the number of locations, the number of polypropylene (PP) curtains, the number of textile (TX) curtains, the date that the curtains were last exchanged, if the location is active (i.e., not blocked or ISO) and if the location includes any textile curtains. A PP curtain is typically single use such that once it has been installed, the curtain should be reinstalled. Textile curtains can be used multiple times. When the textile curtain is removed, it is typically laundered and reinstalled at any location within a facility.

By selecting a particular facility in the list, the system may provide access to more information about each of the locations, users, assigned curtains and protocols associated with that facility. By selecting the locations tab, the system provides a searchable list of the locations. By selecting a particular location, the system provides a location tag code, status indicator, a list of assigned products (with protocol for each of the products) and a list of installed products (including the curtain tag code, installed date and exchange date).

Hospitals and users may not know which type of curtain (e.g., width, height, pattern, material, brand, etc.) should be assigned to a particular location. As such, in various embodiments, the system may also provide a list of available curtains. The available curtains list allows a user to add SKU data, new curtains and/or descriptions of the curtains to the available curtains list. The user may select an available curtain from the available curtains list to assign to a particular location as an assigned product.

In various embodiments, the system may allow some or all of the fields to be edited. For example, a location may be edited to assign new curtains to the location, assign individual protocols to the curtain and to define a location status.

The system may include different types of users. For example, users who are limited to the features which provide the ability to install, remove, and exchange curtains, referred to as Curtain Exchange. Other users who can access the curtain exchange features and also have the ability to record the installation of NFC tags that identify a location within a medical facility, referred to as Location Setup. Both of the user types may have access to multiple facilities. The mobile app may prompt for a facility when needed. By selecting the users tab, the reports, dashboards, inputs and edits may be partially or fully allowed or restricted based on levels of user access and/or user roles. The system may show any of these functions as “active”. The system may allow an administrator to allow or block a function. The system may allow an administrator to allow or block a user from accessing a function or the entire system. For example, an administrator may set an individual user's access to inactive, if the administrator wants to block the individual from accessing a database or if the administrator wants to disable the ability to scan the tags The system also allows authorized users to add a new user with a certain role. The new user may be assigned a user name and password. Such roles may include administrator, staff, distributor staff and distributor administrator. Different roles may be provided different access to certain facilities, certain curtain exchange features, certain tag install features and/or features of the mobile app and web app. The system may also provide the ability to allow access or disallow access to a facility on the system for a particular user.

In various embodiments, the system may store or access a list of all of the curtains in a facility. The curtain data may be manually input or imported from a database or spreadsheet. The system may also prompt the user to enter the curtain details. To help reduce errors, the system may allow the user to select certain protocols and/or curtain types for a subset of curtains. For each curtain, the system may include information about the curtain. The information about each curtain may include the location of the curtain (e.g., 1^{st} Floor, ER01 Bays 1-2), the type of curtain (e.g., 96×36 brown textile), the type of attachment (e.g., snap), the pattern on the curtain (e.g., transparence pattern), the number of curtains in the location and a date that the curtain is scheduled to be replaced.

The system may include a tag. The tag may be in the format of <type>:<date>:<id>. The <type> may include a type of curtain or a location. The <date> may include the date of manufacture of the tag. The <id> may be a UID of the tag (e.g., 14 characters). For quality assurance, the UID may be based on the order date or Purchase Order date. In various embodiments, the tag may include a code, bar code, QR code, RFID NFC and/or any other indicator. The indicator may be configured to be scanned by a scanner (e.g., scanner on a smartphone). Most smartphones include NFC scanning features and functions. Such feature interacts with the antenna of the indicator. Moreover, to increase the reading distance, the system may include combined RFID and NFC technology with a tag having the same UID. Such a combined RFID and NFC technology may enhance the tag reading distance. For example, NFC scans at about 2″ proximity versus RFID that scans at about 10′ proximity.

The system may incorporate cost-efficient RFID devices by using software APIs. The tags may include the new generation tags that “know where they are” that are created by Apple Corporation (may be called “Air Tags”). The new generation tag may allow the staff person to come in and out of the room for regular “protocol” exchanges, but the system would not require any physical input from the staff person. During ISO or other exchanges, the staff person may need to input a reason for the curtain exchange. This new generation tag may be coupled with a raspberry type reader device (instead of the NFC wall tags).

In various embodiments, the tag may include a wall tag that may be located near or inside a location (e.g., room). In response to scanning the wall tag, the system retrieves a list of all curtains within that location. The list of curtains may be retrieved or accessed from a database of curtains. The system may display in a work orders tab each (or a subset) of the curtains and information about each of the curtains such as, for example, location, product type and date exchanged. The work orders tab may also display the number of curtain exchanges that were completed and the number of curtain exchanges that are pending. Such numbers may be for an individual installer, for a particular area of the facility or for the entire facility.

The wall tag is used to verify the location. As such, the use of a wall tag is helpful because the wall tag eliminates or reduces entry errors or mistakes. For example, a worker may mistakenly enter room 120 into a data field, but the worker is installing the curtain in room 102. Moreover, when conducting exchanges under a protocol and/or ISO, the worker only needs to scan the curtain tags once. In particular, the worker needs to only scan the new curtain tags because the scan of the wall tag causes the system to obtain information about all of the prior installed curtains at that location. In other words, the worker does not need to scan each existing curtain tag one-by-one to check out those existing curtains, then scan each new curtain tag to check in the new curtains.

Furthermore, in various embodiments, the wall tag may provide historic detail about a curtain. For example, if a worker scans a curtain tag in order to install that curtain, but that curtain was removed from another location, the system may provide a rejection notice in response to such scan. In particular, the system obtains historical information about the curtain stating that the curtain was previously installed at a first location and removed from that first location. In response to removal of that curtain from the first location, the system associates a code with the removed curtain, wherein the code indicates that such curtain cannot be installed in a second location. The code may indicate that the curtain may never be installed at a new location, or the code may indicate that the curtain must be sterilized before the curtain may be re-installed at a second location or back in the first location.

In various embodiments, the system may also include a curtain tag. The curtain tag may similarly include a code, bar code, QR code, RFID and/or any other indicator. The indicator may be configured to be scanned by a scanner (e.g., scanner on a smartphone). The curtain tag may be located near or on a curtain. The curtain “tag” may not even be a tag at all, but may be incorporated into the curtain material. For example, the laminated (accordia fold) “clean in place” curtain may include laminate that has a “sensor” that could verify “cleaning efficacy.” The verification process may include sending “saturation” data. The laminated material may change color to provide staff a visual affirmation that the staff has sufficiently cleaned and/or treated all surfaces.

In response to scanning the curtain tag, the system retrieves information about that curtain. The information associated with a curtain may include, for example, a curtain tag code, a product description, an exchange date, an install date, a name of the installer, a removal date, a name of the person that removed the curtain, a removal reason, a location of the curtain, a facility where the curtain is located and/or the replacement protocol. Before installing a curtain at a new location (that had been removed from an old location), the staff member scans the curtain tag. If the removal reason associated with the tag includes ISO or COVID, then the system will provide an alert or notification to the staff member that the curtain should not be re-used at the new location. Moreover, the system will restrict the curtain from being entered into the database under the new location.

In response to receiving a selection of the “manage items” tab, in various embodiments, the system displays the options of replace, other or tag info. In response to receiving a selection of the replace option, the system displays a subset or all of the products (e.g., curtain components) at the location. For example, the system may display an entry for a 72×72 blue shower curtain and may display an entry for a 96 in×92 in snap-on mesh having a transparence pattern.

In response to receiving a selection of a particular product in the location, in various embodiments, the system displays options for providing a reason for the removal. For example, the reasons for removal may include ISO, Covid-19, Soiled or Other. In response to receiving a selection of ISO or Covid-19, the system initiates a “replace all” workflow. In various embodiments, the system may determine that an exchange is scheduled based on a protocol exchange, so the selection of a reason for removal may not be required. In other words, the system may direct the worker into the “replace all” workflow, in response to selecting a particular room and/or a particular curtain that was flagged as being impacted by ISO or Covid-19.

In response to receiving a selection of the soiled option, in various embodiments, the system advises that only that particular curtain should be removed and replaced. The worker then scans the curtain tag on that particular curtain that is being removed and replaced. The system then updates the information associated with that particular curtain to show a new date for the next replacement. In response to receiving a selection of the “other” option (under the replace function), the system queries the worker to determine if all curtains in the location should be replaced or if some of the curtains in the location should be replaced. The system may initiate the appropriate workflow based on replacing some or all curtains. The system may display a text box allowing the worker to input an alternative reason for removal. The system updates the information associated with each of the selected curtains with the reason(s) for removal.

In response to receiving a selection of the “other” option (under the manage options function), in various embodiments, the system provides the option to select install or remove. The worker selects install in response to a first time install (e.g., of a new or different curtain type). The worker may also select install to add a curtain (e.g., if the worker forgot to scan a curtain tag). The worker may select remove when the worker is blocking a room for ISO and removing all the curtains from the location. The worker may select remove when the worker is changing a curtain type at the location. For example, the worker may be changing the curtain type from a textile curtain to a recyclable curtain.

In various embodiments, the system may include certain administrative functions. The administrative functions may be available to all users or available only to administrators in response to administrators entering certain verification information. A “manage location” tab may be selected to install or replace a wall tag at a location. In response to receiving an install input, the system may add unconfigured locations or new locations. In response to receiving a new wall tag identifier, the system may prompt the worker to input data to be associated with the new wall tag identifier. In response to receiving a replace input (under the manage location tab), the system may add a wall tag identifier for a wall tag that was removed or lost such that the system associates the existing data associated with the removed or lost wall tag with the new wall tag identifier.

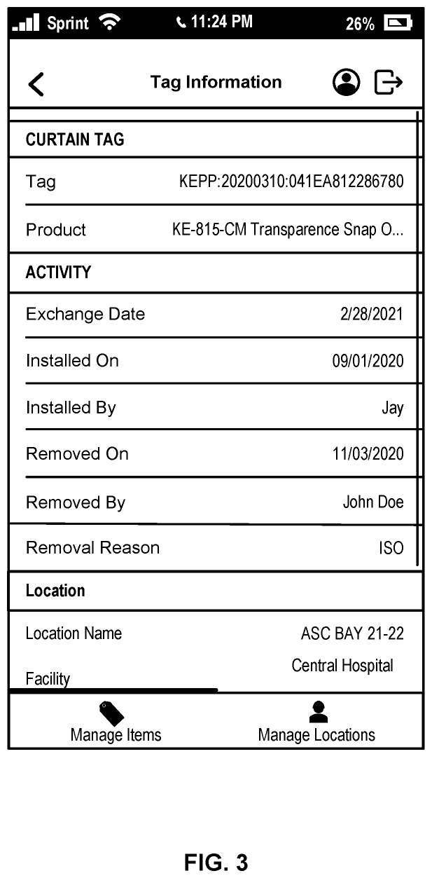

In various embodiments, the system may include a “tag info” button. In response to receiving a selection of the tag info button and a scan of a wall tag or curtain tag, the system may display information about the tag, as set forth in FIG. 3.

In various embodiments, the system may help define protocol compliance for touchpoints (e.g., high risk touch points) in a patient room. The system may also help validate protocol compliance for touch points (e.g., high risk touch points) in a patient room. The system may establish new time-saving protocols, provide software integrated tagging, ensure and validate “clean” touchpoints, use machine learning data analysis and reporting, use API integration to healthcare provider's or manufacturer's systems, and/or integrate blockchain functionality for tamperproof data validation. The system may also be provided via multiple communication protocols including IoT, RFID, Ultra Wideband and Bluetooth 5.0. The system may direct a desired activity into the proper workflow. The system may provide reports about today's exchanges of curtains, upcoming exchanges of curtains and overdue exchanges of curtains. The system may incorporate workflows associated with work orders for exchanging the curtains. As stated above, the system may include a Raspberry type tracking device, so staff only need to verify ISO exchanges. The system may monitor and track the curtains as the curtains are sent to be laundered. Therefore, the system can locate curtains that may become lost in the workflow. The system may also determine a “lifecycle” for the textile curtains. For example, the system may determine that the curtain should be retired after a certain number of laundry cycles and/or a certain level of exposure to detergents. The system may create recycling “green points” reports about the recyclable curtains. The system may incorporate smart surface technology. For example, the system may determine cleaning protocols for certain surfaces and/or validate when such surface is considered to be an acceptable level of clean.

The system may include protocol compliance integrating and/or deploying with UV cleaning equipment. For example, the UV equipment may include a NFC and/or RFID tag that is verified upon entry into the location. The system may monitor the use of the UV equipment over time. The system may integrate with the UV equipment manufacturer's software (e.g., API integration) to help validate cleanliness and/or efficacy.

The detailed description of various embodiments herein makes reference to the accompanying drawings, which show various embodiments by way of illustration. While these various embodiments are described in sufficient detail to enable those skilled in the art to practice the disclosure, it should be understood that other embodiments may be realized and that logical and mechanical changes may be made without departing from the spirit and scope of the disclosure. Thus, the detailed description herein is presented for purposes of illustration only and not of limitation. For example, the steps recited in any of the method or process descriptions may be executed in any order and are not limited to the order presented. Moreover, any of the functions or steps may be outsourced to or performed by one or more third parties. Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set. Furthermore, any reference to singular includes plural embodiments, and any reference to more than one component may include a singular embodiment. Although specific advantages have been enumerated herein, various embodiments may include some, none, or all of the enumerated advantages.

In the detailed description herein, references to “various embodiments,” “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Although the disclosure includes a method, it is contemplated that it may be embodied as computer program instructions on a tangible computer-readable carrier, such as a magnetic or optical memory or a magnetic or optical disk. All structural, chemical, and functional equivalents to the elements of the above-described various embodiments that are known to those of ordinary skill in the art are expressly incorporated herein by reference and are intended to be encompass 140ed by the present claims. Moreover, it is not necessary for a device or method to address each and every problem sought to be solved by the present disclosure, for it to be encompass 140ed by the present claims. Furthermore, no element, component, or method step in the present disclosure is intended to be dedicated to the public regardless of whether the element, component, or method step is explicitly recited in the claims. No claim element is intended to invoke 35 U.S.C. § 112(f) unless the element is expressly recited using the phrase “means for” or “step for”. As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Terms and phrases similar to “associate” and/or “associating” may include tagging, flagging, correlating, using a look-up table or any other method or system for indicating or creating a relationship between elements, such as, for example, (i) a transaction account and (ii) an item (e.g., offer, reward, discount) and/or digital channel. Moreover, the associating may occur at any point, in response to any suitable action, event, or period of time. The associating may occur at pre-determined intervals, periodically, randomly, once, more than once, or in response to a suitable request or action. Any of the information may be distributed and/or accessed via a software enabled link, wherein the link may be sent via an email, text, post, social network input, and/or any other method known in the art.

Computer programs (also referred to as computer control logic) are stored in main memory and/or secondary memory. Computer programs may also be received via communications interface. Such computer programs, when executed, enable the computer system to perform the features as discussed herein. In particular, the computer programs, when executed, enable the processor to perform the features of various embodiments. Accordingly, such computer programs represent controllers of the computer system.

These computer program instructions may be loaded onto a general purpose computer, special purpose computer, or other programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

In various embodiments, software may be stored in a computer program product and loaded into a computer system using a removable storage drive, hard disk drive, or communications interface. The control logic (software), when executed by the processor, causes the processor to perform the functions of various embodiments as described herein. In various embodiments, hardware components may take the form of application specific integrated circuits (ASICs). Implementation of the hardware so as to perform the functions described herein will be apparent to persons skilled in the relevant art(s).

As will be appreciated by one of ordinary skill in the art, the system may be embodied as a customization of an existing system, an add-on product, a processing apparatus executing upgraded software, a stand-alone system, a distributed system, a method, a data processing system, a device for data processing, and/or a computer program product. Accordingly, any portion of the system or a module may take the form of a processing apparatus executing code, an internet based embodiment, an entirely hardware embodiment, or an embodiment combining aspects of the internet, software, and hardware. Furthermore, the system may take the form of a computer program product on a computer-readable storage medium having computer-readable program code means embodied in the storage medium. Any suitable computer-readable storage medium may be utilized, including hard disks, CD-ROM, BLU-RAY DISC®, optical storage devices, magnetic storage devices, and/or the like.

In various embodiments, components, modules, and/or engines of system 100 may be implemented as micro-applications or micro-apps. Micro-apps are typically deployed in the context of a mobile operating system, including for example, a WINDOWS® mobile operating system, an ANDROID® operating system, an APPLE® iOS operating system, a BLACKBERRY® company's operating system, and the like. The micro-app may be configured to leverage the resources of the larger operating system and associated hardware via a set of predetermined rules which govern the operations of various operating systems and hardware resources. For example, where a micro-app desires to communicate with a device or network other than the mobile device or mobile operating system, the micro-app may leverage the communication protocol of the operating system and associated device hardware under the predetermined rules of the mobile operating system. Moreover, where the micro-app desires an input from a user, the micro-app may be configured to request a response from the operating system which monitors various hardware components and then communicates a detected input from the hardware to the micro-app.

The system and method may be described herein in terms of functional block components, screen shots, optional selections, and various processing steps. It should be appreciated that such functional blocks may be realized by any number of hardware and/or software components configured to perform the specified functions. For example, the system may employ various integrated circuit components, e.g., memory elements, processing elements, logic elements, look-up tables, and the like, which may carry out a variety of functions under the control of one or more microprocessors or other control devices. Similarly, the software elements of the system may be implemented with any programming or scripting language such as C, C++, C#, JAVA®, JAVASCRIPT®, JAVASCRIPT® Object Notation (JSON), VB Script, Macromedia COLD FUSION, COBOL, MICROSOFT® company's Active Server Pages, assembly, PERL®, PHP, awk, PYTHON®, Visual Basic, SQL Stored Procedures, PL/SQL, any UNIX® shell script, and extensible markup language (XML) with the various algorithms being implemented with any combination of data structures, objects, processes, routines or other programming elements. Further, it should be noted that the system may employ any number of conventional techniques for data transmission, signaling, data processing, network control, and the like. Still further, the system could be used to detect or prevent security issues with a client-side scripting language, such as JAVASCRIPT®, VBScript, or the like.

The system and method are described herein with reference to screen shots, block diagrams and flowchart illustrations of methods, apparatus, and computer program products according to various embodiments. It will be understood that each functional block of the block diagrams and the flowchart illustrations, and combinations of functional blocks in the block diagrams and flowchart illustrations, respectively, can be implemented by computer program instructions.

Accordingly, functional blocks of the block diagrams and flowchart illustrations support combinations of means for performing the specified functions, combinations of steps for performing the specified functions, and program instruction means for performing the specified functions. It will also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions. Further, illustrations of the process flows and the descriptions thereof may make reference to user WINDOWS® applications, webpages, websites, web forms, prompts, etc. Practitioners will appreciate that the illustrated steps described herein may comprise, in any number of configurations, including the use of WINDOWS® applications, webpages, web forms, popup WINDOWS® applications, prompts, and the like. It should be further appreciated that the multiple steps as illustrated and described may be combined into single webpages and/or WINDOWS® applications but have been expanded for the sake of simplicity. In other cases, steps illustrated and described as single process steps may be separated into multiple webpages and/or WINDOWS® applications but have been combined for simplicity.

In various embodiments, the software elements of the system may also be implemented using a JAVASCRIPT® run-time environment configured to execute JAVASCRIPT® code outside of a web browser. For example, the software elements of the system may also be implemented using NODE.JS® components. NODE.JS® programs may implement several modules to handle various core functionalities. For example, a package management module, such as NPM®, may be implemented as an open source library to aid in organizing the installation and management of third-party NODE.JS® programs. NODE.JS® programs may also implement a process manager, such as, for example, Parallel Multithreaded Machine (“PM2”); a resource and performance monitoring tool, such as, for example, Node Application Metrics (“appmetrics”); a library module for building user interfaces, and/or any other suitable and/or desired module.

Middleware may include any hardware and/or software suitably configured to facilitate communications and/or process transactions between disparate computing systems. Middleware components are commercially available and known in the art. Middleware may be implemented through commercially available hardware and/or software, through custom hardware and/or software components, or through a combination thereof. Middleware may reside in a variety of configurations and may exist as a standalone system or may be a software component residing on the internet server. Middleware may be configured to process transactions between the various components of an application server and any number of internal or external systems for any of the purposes disclosed herein. WEB SPHERE® MQ™ (formerly MQSeries) by IBM®, Inc. (Armonk, N.Y.) is an example of a commercially available middleware product. An Enterprise Service Bus (“ESB”) application is another example of middleware.

The computers discussed herein may provide a suitable website or other internet-based graphical user interface which is accessible by users. In one embodiment, MICROSOFT® company's Internet Information Services (IIS), Transaction Server (MTS) service, and an SQL SERVER® database, are used in conjunction with MICROSOFT® operating systems, WINDOWS NT® web server software, SQL SERVER® database, and MICROSOFT® Commerce Server. Additionally, components such as ACCESS® software, SQL SERVER® database, ORACLE® software, SYBASE® software, INFORMIX® software, MYSQL® software, INTERBASE® software, etc., may be used to provide an Active Data Object (ADO) compliant database management system. In one embodiment, the APACHE® web server is used in conjunction with a LINUX® operating system, a MYSQL® database, and PERL®, PHP, Ruby, and/or PYTHON® programming languages.

For the sake of brevity, conventional data networking, application development, and other functional aspects of the systems (and components of the individual operating components of the systems) may not be described in detail herein. Furthermore, the connecting lines shown in the various figures contained herein are intended to represent exemplary functional relationships and/or physical couplings between the various elements. It should be noted that many alternative or additional functional relationships or physical connections may be present in a practical system.

In various embodiments, the methods described herein are implemented using the various particular machines described herein. The methods described herein may be implemented using the below particular machines, and those hereinafter developed, in any suitable combination, as would be appreciated immediately by one skilled in the art. Further, as is unambiguous from this disclosure, the methods described herein may result in various transformations of certain articles.

In various embodiments, the system and various components may integrate with one or more smart digital assistant technologies. For example, exemplary smart digital assistant technologies may include the ALEXA® system developed by the AMAZON® company, the GOOGLE HOME® system developed by Alphabet, Inc., the HOMEPOD® system of the APPLE® company, and/or similar digital assistant technologies. The ALEXA® system, GOOGLE HOME® system, and HOMEPOD® system, may each provide cloud-based voice activation services that can assist with tasks, entertainment, general information, and more. All the ALEXA® devices, such as the AMAZON ECHO®, AMAZON ECHO DOT®, AMAZON TAP®, and AMAZON FIRE® TV, have access to the ALEXA® system. The ALEXA® system, GOOGLE HOME® system, and HOMEPOD® system may receive voice commands via its voice activation technology, activate other functions, control smart devices, and/or gather information. For example, the smart digital assistant technologies may be used to interact with music, emails, texts, phone calls, question answering, home improvement information, smart home communication/activation, games, shopping, making to-do lists, setting alarms, streaming podcasts, playing audiobooks, and providing weather, traffic, and other real time information, such as news. The ALEXA®, GOOGLE HOME®, and HOMEPOD® systems may also allow the user to access information about eligible transaction accounts linked to an online account across all digital assistant-enabled devices.

The various system components discussed herein may include one or more of the following: a host server or other computing systems including a processor for processing digital data; a memory coupled to the processor for storing digital data; an input digitizer coupled to the processor for inputting digital data; an application program stored in the memory and accessible by the processor for directing processing of digital data by the processor; a display device coupled to the processor and memory for displaying information derived from digital data processed by the processor; and a plurality of databases. Various databases used herein may include: client data; merchant data; financial institution data; and/or like data useful in the operation of the system. As those skilled in the art will appreciate, user computer may include an operating system (e.g., WINDOWS®, UNIX®, LINUX®, SOLARIS®, MACOS®, etc.) as well as various conventional support software and drivers typically associated with computers.

The present system or any part(s) or function(s) thereof may be implemented using hardware, software, or a combination thereof and may be implemented in one or more computer systems or other processing systems. However, the manipulations performed by embodiments may be referred to in terms, such as matching or selecting, which are commonly associated with mental operations performed by a human operator. No such capability of a human operator is necessary, or desirable, in most cases, in any of the operations described herein. Rather, the operations may be machine operations or any of the operations may be conducted or enhanced by artificial intelligence (AI) or machine learning. AI may refer generally to the study of agents (e.g., machines, computer-based systems, etc.) that perceive the world around them, form plans, and make decisions to achieve their goals. Foundations of AI include mathematics, logic, philosophy, probability, linguistics, neuroscience, and decision theory. Many fields fall under the umbrella of AI, such as computer vision, robotics, machine learning, and natural language processing. Useful machines for performing the various embodiments include general purpose digital computers or similar devices.

In various embodiments, the embodiments are directed toward one or more computer systems capable of carrying out the functionalities described herein. The computer system includes one or more processors. The processor is connected to a communication infrastructure (e.g., a communications bus, cross-over bar, network, etc.). Various software embodiments are described in terms of this exemplary computer system. After reading this description, it will become apparent to a person skilled in the relevant art(s) how to implement various embodiments using other computer systems and/or architectures. The computer system can include a display interface that forwards graphics, text, and other data from the communication infrastructure (or from a frame buffer not shown) for display on a display unit.

The computer system also includes a main memory, such as random access memory (RAM), and may also include a secondary memory. The secondary memory may include, for example, a hard disk drive, a solid-state drive, and/or a removable storage drive. The removable storage drive reads from and/or writes to a removable storage unit in a well-known manner. As will be appreciated, the removable storage unit includes a computer usable storage medium having stored therein computer software and/or data.

In various embodiments, secondary memory may include other similar devices for allowing computer programs or other instructions to be loaded into a computer system. Such devices may include, for example, a removable storage unit and an interface. Examples of such may include a program cartridge and cartridge interface (such as that found in video game devices), a removable memory chip (such as an erasable programmable read only memory (EPROM), programmable read only memory (PROM)) and associated socket, or other removable storage units and interfaces, which allow software and data to be transferred from the removable storage unit to a computer system.

The terms “computer program medium,” “computer usable medium,” and “computer readable medium” are used to generally refer to media such as removable storage drive and a hard disk installed in hard disk drive. These computer program products provide software to a computer system.

The computer system may also include a communications interface. A communications interface allows software and data to be transferred between the computer system and external devices. Examples of such a communications interface may include a modem, a network interface (such as an Ethernet card), a communications port, etc. Software and data transferred via the communications interface are in the form of signals which may be electronic, electromagnetic, optical, or other signals capable of being received by communications interface. These signals are provided to communications interface via a communications path (e.g., channel). This channel carries signals and may be implemented using wire, cable, fiber optics, a telephone line, a cellular link, a radio frequency (RF) link, wireless and other communications channels.

As used herein an “identifier” may be any suitable identifier that uniquely identifies an item. For example, the identifier may be a globally unique identifier (“GUID”). The GUID may be an identifier created and/or implemented under the universally unique identifier standard. Moreover, the GUID may be stored as 128-bit value that can be displayed as 32 hexadecimal digits. The identifier may also include a major number, and a minor number. The major number and minor number may each be 16-bit integers.

The firewall may include any hardware and/or software suitably configured to protect CMS components and/or enterprise computing resources from users of other networks. Further, a firewall may be configured to limit or restrict access to various systems and components behind the firewall for web clients connecting through a web server. Firewall may reside in varying configurations including Stateful Inspection, Proxy based, access control lists, and Packet Filtering among others. Firewall may be integrated within a web server or any other CMS components or may further reside as a separate entity. A firewall may implement network address translation (“NAT”) and/or network address port translation (“NAPT”). A firewall may accommodate various tunneling protocols to facilitate secure communications, such as those used in virtual private networking. A firewall may implement a demilitarized zone (“DMZ”) to facilitate communications with a public network such as the internet. A firewall may be integrated as software within an internet server or any other application server components, reside within another computing device, or take the form of a standalone hardware component.

Any databases discussed herein may include relational, hierarchical, graphical, blockchain, object-oriented structure, and/or any other database configurations. Any database may also include a flat file structure wherein data may be stored in a single file in the form of rows and columns, with no structure for indexing and no structural relationships between records. For example, a flat file structure may include a delimited text file, a CSV (comma-separated values) file, and/or any other suitable flat file structure. Common database products that may be used to implement the databases include DB2® by IBM® (Armonk, N.Y.), various database products available from ORACLE® Corporation (Redwood Shores, Calif.), MICROSOFT ACCESS® or MICROSOFT SQL SERVER® by MICROSOFT® Corporation (Redmond, Wash.), MYSQL® by MySQL AB (Uppsala, Sweden), MONGODB®, Redis, APACHE CASSANDRA®, HBASE® by APACHE®, MapR-DB by the MAPR® corporation, or any other suitable database product. Moreover, any database may be organized in any suitable manner, for example, as data tables or lookup tables. Each record may be a single file, a series of files, a linked series of data fields, or any other data structure.

As used herein, big data may refer to partially or fully structured, semi-structured, or unstructured data sets including millions of rows and hundreds of thousands of columns. A big data set may be compiled, for example, from a history of purchase transactions over time, from web registrations, from social media, from records of charge (ROC), from summaries of charges (SOC), from internal data, or from other suitable sources. Big data sets may be compiled without descriptive metadata such as column types, counts, percentiles, or other interpretive-aid data points.

Association of certain data may be accomplished through any desired data association technique such as those known or practiced in the art. For example, the association may be accomplished either manually or automatically. Automatic association techniques may include, for example, a database search, a database merge, GREP, AGREP, SQL, using a key field in the tables to speed searches, sequential searches through all the tables and files, sorting records in the file according to a known order to simplify lookup, and/or the like. The association step may be accomplished by a database merge function, for example, using a “key field” in pre-selected databases or data sectors. Various database tuning steps are contemplated to optimize database performance. For example, frequently used files such as indexes may be placed on separate file systems to reduce In/Out (“I/O”) bottlenecks.

More particularly, a “key field” partitions the database according to the high-level class of objects defined by the key field. For example, certain types of data may be designated as a key field in a plurality of related data tables and the data tables may then be linked on the basis of the type of data in the key field. The data corresponding to the key field in each of the linked data tables is preferably the same or of the same type. However, data tables having similar, though not identical, data in the key fields may also be linked by using AGREP, for example. In accordance with one embodiment, any suitable data storage technique may be utilized to store data without a standard format. Data sets may be stored using any suitable technique, including, for example, storing individual files using an ISO/IEC 7816-4 file structure; implementing a domain whereby a dedicated file is selected that exposes one or more elementary files containing one or more data sets; using data sets stored in individual files using a hierarchical filing system; data sets stored as records in a single file (including compression, SQL accessible, hashed via one or more keys, numeric, alphabetical by first tuple, etc.); data stored as Binary Large Object (BLOB); data stored as ungrouped data elements encoded using ISO/IEC 7816-6 data elements; data stored as ungrouped data elements encoded using ISO/IEC Abstract Syntax Notation (ASN.1) as in ISO/IEC 8824 and 8825; other proprietary techniques that may include fractal compression methods, image compression methods, etc.

In various embodiments, the ability to store a wide variety of information in different formats is facilitated by storing the information as a BLOB. Thus, any binary information can be stored in a storage space associated with a data set. As discussed above, the binary information may be stored in association with the system or external to but affiliated with the system. The BLOB method may store data sets as ungrouped data elements formatted as a block of binary via a fixed memory offset using either fixed storage allocation, circular queue techniques, or best practices with respect to memory management (e.g., paged memory, least recently used, etc.). By using BLOB methods, the ability to store various data sets that have different formats facilitates the storage of data, in the database or associated with the system, by multiple and unrelated owners of the data sets. For example, a first data set which may be stored may be provided by a first party, a second data set which may be stored may be provided by an unrelated second party, and yet a third data set which may be stored may be provided by a third party unrelated to the first and second party. Each of these three exemplary data sets may contain different information that is stored using different data storage formats and/or techniques. Further, each data set may contain subsets of data that also may be distinct from other subsets.

As stated above, in various embodiments, the data can be stored without regard to a common format. However, the data set (e.g., BLOB) may be annotated in a standard manner when provided for manipulating the data in the database or system. The annotation may comprise a short header, trailer, or other appropriate indicator related to each data set that is configured to convey information useful in managing the various data sets. For example, the annotation may be called a “condition header,” “header,” “trailer,” or “status,” herein, and may comprise an indication of the status of the data set or may include an identifier correlated to a specific issuer or owner of the data. In one example, the first three bytes of each data set BLOB may be configured or configurable to indicate the status of that particular data set; e.g., LOADED, INITIALIZED, READY, BLOCKED, REMOVABLE, or DELETED. Subsequent bytes of data may be used to indicate for example, the identity of the issuer, user, transaction/membership account identifier or the like. Each of these condition annotations are further discussed herein.

The data set annotation may also be used for other types of status information as well as various other purposes. For example, the data set annotation may include security information establishing access levels. The access levels may, for example, be configured to permit only certain individuals, levels of employees, companies, or other entities to access data sets, or to permit access to specific data sets based on the transaction, merchant, issuer, user, or the like. Furthermore, the security information may restrict/permit only certain actions, such as accessing, modifying, and/or deleting data sets. In one example, the data set annotation indicates that only the data set owner or the user are permitted to delete a data set, various identified users may be permitted to access the data set for reading, and others are altogether excluded from accessing the data set. However, other access restriction parameters may also be used allowing various entities to access a data set with various permission levels as appropriate.

The data, including the header or trailer, may be received by a standalone interaction device configured to add, delete, modify, or augment the data in accordance with the header or trailer. As such, in one embodiment, the header or trailer is not stored on the transaction device along with the associated issuer-owned data, but instead the appropriate action may be taken by providing to the user, at the standalone device, the appropriate option for the action to be taken. The system may contemplate a data storage arrangement wherein the header or trailer, or header or trailer history, of the data is stored on the system, device or transaction instrument in relation to the appropriate data.

One skilled in the art will also appreciate that, for security reasons, any databases, systems, devices, servers, or other components of the system may consist of any combination thereof at a single location or at multiple locations, wherein each database or system includes any of various suitable security features, such as firewalls, access codes, encryption, decryption, compression, decompression, and/or the like.

Practitioners will also appreciate that there are a number of methods for displaying data within a browser-based document. Data may be represented as standard text or within a fixed list, scrollable list, drop-down list, editable text field, fixed text field, pop-up window, and the like. Likewise, there are a number of methods available for modifying data in a web page such as, for example, free text entry using a keyboard, selection of menu items, check boxes, option boxes, and the like.

The data may be big data that is processed by a distributed computing cluster. The distributed computing cluster may be, for example, a HADOOP® software cluster configured to process and store big data sets with some of nodes comprising a distributed storage system and some of nodes comprising a distributed processing system. In that regard, distributed computing cluster may be configured to support a HADOOP® software distributed file system (HDFS) as specified by the Apache Software Foundation at www.hadoop.apache.org/docs.

As used herein, the term “network” includes any cloud, cloud computing system, or electronic communications system or method which incorporates hardware and/or software components. Communication among the parties may be accomplished through any suitable communication channels, such as, for example, a telephone network, an extranet, an intranet, internet, point of interaction device (point of sale device, personal digital assistant (e.g., an IPHONE® device, a BLACKBERRY® device), cellular phone, kiosk, etc.), online communications, satellite communications, off-line communications, wireless communications, transponder communications, local area network (LAN), wide area network (WAN), virtual private network (VPN), networked or linked devices, keyboard, mouse, and/or any suitable communication or data input modality. Moreover, although the system is frequently described herein as being implemented with TCP/IP communications protocols, the system may also be implemented using IPX, APPLETALK® program, IP-6, NetBIOS, OSI, any tunneling protocol (e.g. IPsec, SSH, etc.), or any number of existing or future protocols. If the network is in the nature of a public network, such as the internet, it may be advantageous to presume the network to be insecure and open to eavesdroppers. Specific information related to the protocols, standards, and application software utilized in connection with the internet is generally known to those skilled in the art and, as such, need not be detailed herein.

“Cloud” or “Cloud computing” includes a model for enabling convenient, on-demand network access to a shared pool of configurable computing resources (e.g., networks, servers, storage, applications, and services) that can be rapidly provisioned and released with minimal management effort or service provider interaction. Cloud computing may include location-independent computing, whereby shared servers provide resources, software, and data to computers and other devices on demand.

As used herein, “transmit” may include sending electronic data from one system component to another over a network connection. Additionally, as used herein, “data” may include encompassing information such as commands, queries, files, data for storage, and the like in digital or any other form.

Any database discussed herein may comprise a distributed ledger maintained by a plurality of computing devices (e.g., nodes) over a peer-to-peer network. Each computing device maintains a copy and/or partial copy of the distributed ledger and communicates with one or more other computing devices in the network to validate and write data to the distributed ledger. The distributed ledger may use features and functionality of blockchain technology, including, for example, consensus-based validation, immutability, and cryptographically chained blocks of data. The blockchain may comprise a ledger of interconnected blocks containing data. The blockchain may provide enhanced security because each block may hold individual transactions and the results of any blockchain executables. Each block may link to the previous block and may include a timestamp. Blocks may be linked because each block may include the hash of the prior block in the blockchain. The linked blocks form a chain, with only one successor block allowed to link to one other predecessor block for a single chain. Forks may be possible where divergent chains are established from a previously uniform blockchain, though typically only one of the divergent chains will be maintained as the consensus chain. In various embodiments, the blockchain may implement smart contracts that enforce data workflows in a decentralized manner. The system may also include applications deployed on user devices such as, for example, computers, tablets, smartphones, Internet of Things devices (“IoT” devices), etc. The applications may communicate with the blockchain (e.g., directly or via a blockchain node) to transmit and retrieve data. In various embodiments, a governing organization or consortium may control access to data stored on the blockchain. Registration with the managing organization(s) may enable participation in the blockchain network.

Data transfers performed through the blockchain-based system may propagate to the connected peers within the blockchain network within a duration that may be determined by the block creation time of the specific blockchain technology implemented. For example, on an ETHEREUM®-based network, a new data entry may become available within about 13-20 seconds as of the writing. On a HYPERLEDGER® Fabric 1.0 based platform, the duration is driven by the specific consensus algorithm that is chosen, and may be performed within seconds. In that respect, propagation times in the system may be improved compared to existing systems, and implementation costs and time to market may also be drastically reduced. The system also offers increased security at least partially due to the immutable nature of data that is stored in the blockchain, reducing the probability of tampering with various data inputs and outputs. Moreover, the system may also offer increased security of data by performing cryptographic processes on the data prior to storing the data on the blockchain. Therefore, by transmitting, storing, and accessing data using the system described herein, the security of the data is improved, which decreases the risk of the computer or network from being compromised.

In various embodiments, the system may also reduce database synchronization errors by providing a common data structure, thus at least partially improving the integrity of stored data. The system also offers increased reliability and fault tolerance over traditional databases (e.g., relational databases, distributed databases, etc.) as each node operates with a full copy of the stored data, thus at least partially reducing downtime due to localized network outages and hardware failures. The system may also increase the reliability of data transfers in a network environment having reliable and unreliable peers, as each node broadcasts messages to all connected peers, and, as each block comprises a link to a previous block, a node may quickly detect a missing block and propagate a request for the missing block to the other nodes in the blockchain network.

The particular blockchain implementation described herein provides improvements over conventional technology by using a decentralized database and improved processing environments. In particular, the blockchain implementation improves computer performance by, for example, leveraging decentralized resources (e.g., lower latency). The distributed computational resources improves computer performance by, for example, reducing processing times. Furthermore, the distributed computational resources improves computer performance by improving security using, for example, cryptographic protocols.

Any communication, transmission, and/or channel discussed herein may include any system or method for delivering content (e.g. data, information, metadata, etc.), and/or the content itself. The content may be presented in any form or medium, and in various embodiments, the content may be delivered electronically and/or capable of being presented electronically. For example, a channel may comprise a website, mobile application, or device (e.g., FACEBOOK®, YOUTUBE®, PANDORA®, APPLE TV®, MICROSOFT® XBOX®, ROKU®, AMAZON FIRE®, GOOGLE CHROMECAST™, SONY® PLAYSTATION®, NINTENDO® SWITCH®, etc.) a uniform resource locator (“URL”), a document (e.g., a MICROSOFT® Word or EXCEL™, an ADOBE® Portable Document Format (PDF) document, etc.), an “ebook,” an “emagazine,” an application or microapplication (as described herein), an short message service (SMS) or other type of text message, an email, a FACEBOOK® message, a TWITTER® tweet, multimedia messaging services (MMS), and/or other type of communication technology. In various embodiments, a channel may be hosted or provided by a data partner. In various embodiments, the distribution channel may comprise at least one of a merchant website, a social media website, affiliate or partner websites, an external vendor, a mobile device communication, social media network, and/or location based service. Distribution channels may include at least one of a merchant website, a social media site, affiliate or partner websites, an external vendor, and a mobile device communication. Examples of social media sites include FACEBOOK®, FOURSQUARE®, TWITTER®, LINKEDIN®, INSTAGRAM®, PINTEREST®, TUMBLR®, REDDIT®, SNAPCHAT®, WHATSAPP®, FLICKR®, VK®, QZONE®, WECHAT®, and the like. Examples of affiliate or partner websites include AMERICAN EXPRESS®, GROUPON®, LIVINGSOCIAL®, and the like. Moreover, examples of mobile device communications include texting, email, and mobile applications for smartphones.

What is claimed is:

1. A method comprising:
receiving, by the processor, a scan of a wall tag associated with a location;
providing, by the processor, a list of installed curtains in the location;
receiving, by the processor, a replacement reason for one or more of the installed curtains; and
receiving, by the processor, a scan of a curtain tag for a new curtain.

2. The method of claim 1, further comprising providing a replace all notification, in response to receiving a Covid-19 reason for removal.

3. The method of claim 1, further comprising:
receiving, by the processor, a work order request; and
providing, by the processor, a list of scheduled curtain exchanges.

4. The method of claim 1, further comprising providing a notification that the new curtain cannot be installed, in response to the new curtain being from a previous location with a health concern.

5. The method of claim 1, further comprising receiving, by the processor, a scan of the curtain tag for the installed curtain that is being removed, in response to the installed curtain being soiled.

6. The method of claim 1, further comprising providing, by the processor, an option to replace all or replace some, in response to another reason for replacing the installed curtain.

7. The method of claim 1, further comprising receiving, by the processor, an install command, in response to a curtain being added without the curtain tag being scanned.

8. The method of claim 1, further comprising receiving, by the processor, a remove command, in response to a room being blocked for ISO and no curtains remain at the location.

9. The method of claim 1, further comprising receiving, by the processor, a remove command, in response to changing the installed curtains to a different curtain type at the location.

10. The method of claim 1, wherein the replacement reasons include at least one of ISO, Covid-19, Soiled or Other.

11. The method of claim 1, wherein a replacement protocol is associated with each of the installed curtains.

12. The method of claim 1, wherein a replacement protocol is associated with each of the installed curtains, wherein the replacement protocol is at least one of per patient, weekly, monthly, quarterly, bi-annually or annually.

13. The method of claim 1, further comprising providing, by the processor, a report about curtain exchanges.

14. The method of claim 1, further comprising providing, by the processor, a report about overdue, upcoming, ISO trending or historical curtain exchanges.
